(19) United States
(12) Patent Application Publication
(10) Pub. No.: US 2004/0191334 A1
(43) Pub. Date: Sep. 30, 2004

(54) Use of transhinone derivates as cholinesterase inhibitors in treating related diseases

(76) Inventors: Pang-Chui Shaw, Hong Kong (CN); Michelle Tsz-Wan Ho, Hong Kong (CN); Kwok-Pui Fung, Hong Kong (CN); David Chi-Cheong Wan, Hong Kong (CN)

Correspondence Address: MCANDREWS HELD & MALLOY, LTD, 500 WEST MADISON STREET, SUITE 3400, CHICAGO, IL, 60661

(21) Appl. No.: 10/396,862

(22) Filed: Mar. 24, 2003

(51) Int. Cl. A61K 35/78; A61K 31/12

(52) U.S. Cl. 424/746; 514/675

(57) ABSTRACT

This present invention provides a new use of tanshinone derivatives as acetylcholinesterase inhibitors and in treating diseases associated with the depletion of acetylcholine such as cognitive impairment including Alzheimer's disease, insomnia, vascular dementia, memory loss, disorders of attention and other sleeping disorders. The active compounds commonly found in the root of Salvia genus, particularly Salvia miltiorrhiza, Danshen.

### FIELD OF THE INVENTION

The present invention provides a novel use of tanshinone derivates as acetylcholinesterase inhibitors and in treating diseases associated with the depletion of acetylcholine such as cognitive impairment.

### BACKGROUND OF THE INVENTION

Alzheimer's disease (AD) is a neurodegenerative disease, affecting 11% and 50% of the population over 65 and 85, respectively. At present, there is still no cure for AD. One of the neuropathology outflows of AD is the depletion of a major neurotransmitter in the basal forebrain —acetylcholine. At present, the only drug FDA approved for treating AD is based on its acetylcholinestease (AChE) inhibitory activities, thereby restoring the acetylcholine level at the synapses.

Acetylcholine (ACh) is one of the many neurotransmitters in the brain and is also an important neurotransmitter at the neuromuscular junction. It also mediates certain parasympathetic nervous system and leads to the contraction of smooth muscle, vasodilation, increases secretion and lowers the heart rate. ACh can react not only nicotinic but also muscarinic receptors.

In the case of Parkinson's patients, there is always a balance problem between dopamine and acetylcholine level. The depletion of dopamine always leads to relative increase in acetylcholine level. The over-stimulation of cholinergic receptors in the neuromuscular junction results in the shaking of hands or impaired body postulation in such patients.

ACh plays an important role for memory and cognitive acquisition, and depletion of ACh will result in loss of memory and the ability to perform daily routine. Alzheimer's disease is characterized as a disease of cholinergic deficit in the brain.

Rapid eye movement behavior disorder (RBD) and its related symptoms are markedly improved by the treatment with an AChE inhibitor, Donepezil.

Acetylcholinesterase (AChE) is responsible for the degradation of ACh.

The type of inhibition caused by acetylcholinesterase can be presented in several forms, either by directly binding to the active center gorge or to the allosteric modulating sites.

The active site of AChE is buried deep down at the center gorge, with a 20 angstroms along the pathway from the surface of the enzyme.

Butyrylcholinesterase (BuChE) is abundantly found in plasma. It can hydrolyse acetylcholine in the neuromuscular junction, performing the same task as that of AChE. Non-specific inhibition of BuChE in conjunction with AChE inhibition always leads to neurotoxicity.

AChE inhibitors are an important group of drugs to treat Alzheimer's disease.

Four drugs as AChE inhibitors have been approved by FDA for the treatment of AD. These include Donepezil (Aricept™), ENA-713 (Exelon™), Galantamine (Reminyl™) and Tacrine (Cognex™).

A survey of Traditional Chinese Medicinal formulas with hundreds of years of clinical experience shows that Salvia miltiorrhiza is frequently prescribed as remedies capable of easing the mind and among other pharmacological properties such as promoting blood flow, stimulating menstrual discharge, menstrual disorder, dysmenorrhea and relieving pain.

In the prior art in practising Chinese medicine, Danshen, the root of Salvia miltiorrhiza, is a component of the prescription, “Tianwang Buxin Dan”. The prescription is mainly used for treatment of insomnia, metal agitation and memory problem. A very small amount of Danshen is found in the prescription. Danshen is seldom prescribed as a single herb. The function of Danshen in this prescription is primarily for “heart nourishing and spirit pacifying action”, as described. (Tang W, Eisenbrand G, Chinese Drugs of Plant Origin, Springer-Verlag Berlin Heidelberg 1992.)

In modern Chinese medicine, Danshen is recognized as a useful blood vitalizing agent. Salvia miltiorrhiza is able to increase coronary flow and reduce coronary resistance, and to produce vasodilation action. It can also possess anti-platelet aggregation and sedative effect. (Tang W, Eisenbrand G, Chinese Drugs of Plant Origin, Springer-Verlag Berlin Heidelberg 1992,

[symbol]

[symbol]

[symbol]

[symbol]

Danshen is also recommended for prevention of liver damage due to viral hepatitis and also fibrosis of liver. (Tang W, Eisenbrand G, Chinese Drugs of Plant Origin, Springer-Verlag Berlin Heidelberg 1992.)

Due to the blood vitalizing property, Danshen is also used for treatment of menstrual disorder, menostasis, menorrrhalgia.

[symbol]

[symbol]

[symbol]

[symbol]

The injection of Danshen increases the flow rate in the aorta and the circumflex branch of the left coronary artery (Yang et al, Studies on Chinese Patent 1979 Medicine (1): 8)

Rabbits or rats with acute myocardial ischemia induced by pituitrin could be improved or counteracted by treatment with Danshen. (Chemistry of Nature Drugs Section, Faculty of Pharmacy, 1980)

Danshen can increase the coronary flow rate and be shown to have a protective effect on heart against ischemia and reperfusion. (Zhou W et al., Protective effect of danshen during myocardial ischemia and reperfusion; an isolated rat heart study. Am J Chin Med 1990; 18(1-2):19-24)

Danshen has recently been shown to attenuate intimal thickening in the balloon-injured abdominal aorta of cholesterol-fed rabbits. (Chen. Y L. et al., Salvia miltiorrhiza inhibits intimal hyperplasia and monocyte chemotactic protein-1 expression after balloon injury in cholesterol-fed rabbits., J Cell Biochem. 83:484-493, 2001)

Recent study has shown that, magnesium tanshinoate B (MTB), an active compound from Danshen, can inhibit the oxidative modification of low density lipoprotein (LDL). (Karmiin O. et al., Magnesium tanshinoate B (MTB) inhibits low density lipoprotein oxidation. Life Sci 2001 Jan 12,68(8):903-12)

Hot water extract of Salvia Miltiorrhiza has anti-fibrotic effects on liver fibrosis induced rats with a marked increase in concentrations of aspartate transaminase, alanine transaminase, alkaline phosphatase, total bilirubin and total cholesterol level. (Nan J. X. et al., Anti-fibrotic effects of a hot-water extract from Salvia Miltiorrhiza root on liver fibrosis induced by biliary obstruction in rats. J Pharm Pharmacol. 2001 Feb; 53(2):197-204.)

It is found that cryptotanshinone and dihydrotanshinone I had antibacterial activity against a broad range of Gram positive bacteria. (Lee D. S. et al., Antibacterial activities of cryptotanshinone and dihydrotanshinone I from a medicinal herb, Salvia miltiorrhiza Bunge. Biosci Biotechnol Biochem 1999 Dec;63(12): 2236-9)

Immunologically, dihydrotanshinone was shown to effectively inhibit interleukin-12 (IL-12) and interferon-gamma (IFN-gamma) production when compared with cryptotanshinone and tanshinone I. (Kang B. Y et al., Inhibition of interleukin-12 and interferon-gamma production in immune cells by tanshinones from Salvia miltiorrhiza. Immunopharmacology. 2000 Sep; 49(3):355-61)

The anti-cholinesterase activity of Salvia Lavandulaefolia was recently demonstrated. Unlike our claims, the inhibition was proposed to be contributed by monoterpenoid (camphor and 1,8-cineole). (Perry N S, Houghton P J, Theobald A, Jenner P, Perry E K, In-vitro inhibition of human erythrocyte acetylcholinesterase by salvia lavandulaefolia essential oil and constituent terpenes. J Pharm Pharmacol. 2000 Jul;52(7):895-902.)

Salvia officinalis extract was found to be effective in treating mild to moderate Alzheimer's disease in a small-scale clinical trial. (Akhondzadeh S, Noroozian M, Mohammadi M, Ohadinia S, Jamshidi A H, Khani M, Salvia officinalis extract in the treatment of patients with mild to moderate Alzheimer's disease: a double blind, randomized and placebo-controlled trial. J Clin Pharm Ther. 2003 Feb, 28(1):53-59.)

### SUMMARY OF THE INVENTION

Therefore, an object of the present invention is to provide a pharmaceutical composition for treating diseases or disorders associated with cholinesterases, particularly deficit of acetylcholine, comprising a therapeutically effective amount of an active component selected from a compound of cryptotanshinone, dihyrotanshinone I, tanshinone I, or a pharmaceutically acceptable salt thereof, and a mixture of the compounds or their pharmaceutically acceptable salts or both, and a pharmaceutically acceptable carrier.

Another object of the present invention is to provide a use of a compound of cryptotanshinone, dihyrotanshinone I, tanshinone I, or a pharmaceutically acceptable salt thereof, and a mixture of the compounds or their pharmaceutically acceptable salts or both in preparing a medicament for treating diseases or disorders associated with deficit of acetylcholine.

Still another object of the present invention is to provide a method for treating diseases or disorders associated with deficit of acetylcholine comprising administrating to a patient a therapeutically effective amount of an active component selected from a compound of cryptotanshinone, dihyrotanshinone I, tanshinone I, or a pharmaceutically acceptable salt thereof, and a mixture of the compounds or their pharmaceutically acceptable salts or both.

### BRIEF DESCRIPTION OF THE DRAWINGS

FIG. 1 is a graph showing hAChE inhibition of the aqueous and ethanol extract of the root of Salvia miltiorrhiza.

FIG. 2 shows inhibition of selected active components from Danshen on hAChE.

FIG. 3 shows inhibition of selected active components from Salvia on hAChE.

FIG. 4a shows inhibition of cryptotanshine on hAChE.and hBChE.

FIG. 4b shows inhibition of dihydrotanshinone on hAChE.and hBChE.

FIG. 5a shows MTT cytotoxicity of active components according to the invention on neuronal cell line-SHSY5Y

FIG. 5b shows MTT cytotoxicity of active components according to the invention on mouse neuronal cell line NIE-115.

### DETAILED DESCRIPTION OF THE INVENTION

The pharmaceutical composition of the invention for treating diseases or disorders associated with cholinesterases comprises a therapeutically effective amount of an active component selected from a compound of cryptotanshinone, dihydrotanshinone I, tanshinone I, or a pharmaceutically acceptable salt thereof, and a mixture of the compounds or their pharmaceutically acceptable salts or both, and a pharmaceutically acceptable carrier.

Tanshinone derivatives existing in Danshen include cryptoanshinone, dihydrotanshinone tanshinone I, and tanshinone II, which have the following chemical structures. However, in the invention, the active compound excludes tanshinnone II.

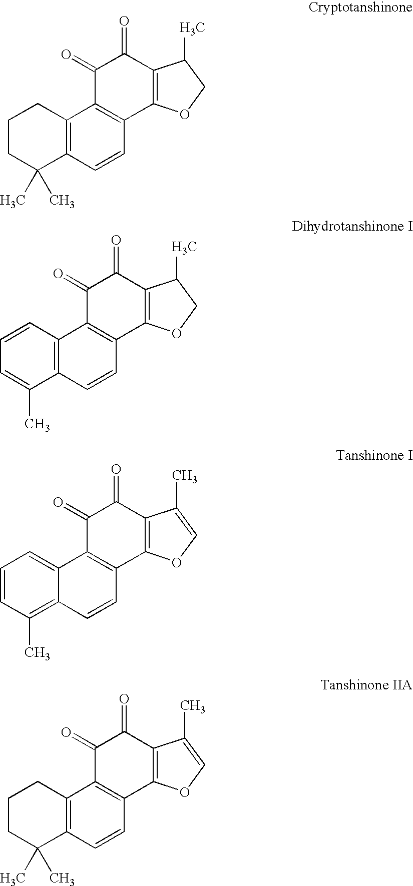

The active compound of tanshinone shows a weak acidity. In the present invention, therefore, the term “pharmaceutically acceptable salt” is intended to mean those alkali metal salts such as a sodium or potassium salt, alkaline earth metal salts such as a calcium or magnesium salt, organic amine salts such as a salt with trimethylamine, triethylamine, pyridine, picoline, dicyclohexylamine, or N,N′-dibenzylethylenediamine.

In the invention, the term “effective compound” or “effective ingredient” or “effective component” used refers to the active tanshinone compound or salt thereof that is mentioned above or a mixture of both, or an extract of a plant of Salvia genus.

The plant in the invention is preferably selected from Salvia miltiorrhiza, Salvia lavandulaefolia, Salvia divinorum and Salvia officinalis. More preferably, the plant used in the invention is Salvia miltiorrhiza. The extract of the plant preferably comes from the root of the plant.

The extract of the plant includes aqueous extracts and C₁₋₄ alkanol extracts. The hot water extract and C₁₋₄ alkanol extract are preferable. The ethanol extract is most preferable.

The term “therapeutically effective amount” or “effective amount” used herein is intended to mean an amount of the active component effective to achieve its intended purposes. The dose will vary depending upon the symptoms, sex, age, and weight of patients, method of administration, time and intervals of administration and properties, dispensing, and kind of pharmaceutical formulations, specific effective ingredients, etc. It is appreciated for those skilled in the art that there is no particular limitation with respect to the dose. Normally the active component may be administered in a dose of about 0.005 to 500 mg, preferably 0.1 to 300 mg, more preferably 1 to 100 mg, per day per patient, ordinarily in one to four portions. However, in most instances, an effective daily dosage will be in the range of from about 0.05 mg/kg to about 25 mg/kg of body weight, and preferably, of from 0.1 mg/kg to about 10 mg/kg of body weight, administered in single or divided doses. In some cases, however, it may be necessary to use dosages outside these limits, which will be determined by the prescribing physician

Pharmaceutical compositions for administration according to the present invention can comprise at least one active compound in a pharmaceutically acceptable form optionally combined with a pharmaceutically acceptable carrier.

The term “pharmaceutically acceptable carrier” used herein means excipients and auxiliaries which facilitate processing of the active component into formulations which can be used pharmaceutically. The formulations can be administered orally, intramuscularly, intraperitoneally, subcutaneously and intravenously. Preferably, the formulations, particularly those such as tablets, dragees, troches and capsules, as well as suitable solutions, contain from about 0.01 to 99.99 percent by weight, preferably from about 25 to 75 percent by weight of active component(s) together with the excipient and/or auxiliary.

Suitable excipients used in the invention includes fillers such as saccharides, for example, lactose or sucrose, mannitol or sorbitol; cellulose derivatives; magnesium sulfate; calcium phosphates such as tricalcium phosphate or calcium hydrogen phosphate; as well as binder such as starch paste, for example, maize starch, wheat starch, rice starch, potato starch; gelatin; tragacanth; and/or polyvinylpyrrolidone.

Suitable auxiliaries that may be used in the invention include flow-regulating agents and lubricants, such as talc, silica, stearic acid or salts thereof (such as magnesium stearate), and/or polyethylene glycol. Dragee cores are provided with suitable coatings which, if desired, are resistant to gastric juices. For this purpose, concentrated saccharide solutions can be used, which can optionally contain gum arabic, talc, polyvinyl pyrrolidione, polyethylene glycol and/or titanium dioxide, lacquer solutions and suitable organic solvents or solvent mixtures. In order to produce coatings resistant to gastric juices, i.e., enteric coatings, solutions of suitable cellulose preparations such as acetylcellulose phthalate or hydroxypropylmethyl cellulose phthalate are used. Dyestuffs or pigments can be added to the tablets or dragee coatings.

The composition of the present invention may be formulated in the form of injections, such as intravenous, subcutaneous, and intramuscular injections, suppositories, or sublingual tablets. Pharmaceutical formulations in the dosage form of, e.g., injections, suppositories, sublingual tablets, tablets, and capsules are prepared according to methods which are commonly accepted in the art.

In preparing injections, the effective ingredient is blended, if necessary, with a pH modifier, a buffer, a solubilizing agent, a suspending agent, a stabilizer, and a preservative, followed by preparation of an intravenous, subcutaneous, or intramuscular injection according to an ordinary method.

Examples of the solubilizing agent include polyoxyethylene hydrogenated castor oil, polysorbate 80, nicotinamide, polyoxyethylene sorbitan monolaurate, macrogol, and an ethyl ester of castor oil fatty acid. Examples of the suspending agents include methylcellulose, polysorbate 80, hydroxyethylcellulose, acacia, powdered tragacanth, sodium carboxymethylcellulose, and polyoxyethylene sorbitan monolaurate.

Examples of the stabilizer include sodium sulfite, sodium metasulfite, and ether, and examples of the preservative include methyl p-hydroxybenzoate, ethyl p-hydroxybenzoate, sorbic acid, phenol, cresol, and chlorocresol.

In the invention, when the active compound or the composition is administered orally, it can be in the form of tablets or capsules, or as an aqueous solution or suspension. In the case of tablets, carriers which are commonly used include lactose, mannitol and corn starch, and lubricating agents, such as magnesium stearate, are commonly added. In the case of the capsule form, the active compound can be administered in dry form in a hard gelatin capsule or in a suitable gelled or liquid vehicle, such as a liquid polyethylene glycol or a carrageenan gel, in a soft gelatin capsule. When aqueous suspensions are required for oral use, the active ingredient is combined with emulsifying and suspending agents. If desired, certain sweetening and/or flavoring agents may be added.

The diseases or disorders the active ingredient of the invention treats are intended to mean those associated with cholinesterase. In the invention, the diseases or disorders includes those of cognitive impairment such as presenile dementia, insomnia and Alzheimer's disease. The examples of disorders according to the invention include attention defective disorder, attention defective disorder, vascular dementia, rapid eye movement behavior disorder (RBD).

### EXAMPLE b 1

Preparation of an Aqueous Extract of the Plant of Danshen

10 gram of Danshen from Hibei was commercially obtained from the Tongrentang Hong Kong Ltd. The root of Salvia miltiorrhiza, was boiled in 80 ml of distilled water under reflux for 45 minutes. The boiling extract was then filtered and the clear filtrate is then subject to lyophilization. The lyophilized material that is in powder form was weighted (2 g).

### EXAMPLE 2

Preparation of an Ethanol Extract of the Plant of Danshen

10 gram of the dried Danshen was soaked in 50 ml of absolute ethanol overnight. The unfiltered extract was stored and the whole process was repeated (i.e. use another 50 mL absolute ethanol to re-soak the herb for a second time). A total of 100 mL extract were then cleared by filtration on a 3M paper and then subject to lyophillization. The lyophilized material that is in sticky jelly form was weighted (100 mg). EXAMPLE 3

| | |
|---|---|
| Cryptotanshinone | 0.01 g |
| Corn starch | 95.99 g |
| Magnesium stearate | 4 g |

All ingredients were mixed uniformly and were formed as 100 tablets.

### EXAMPLE 4

| | | |
|---|---|---|
| Extract obtained in Example 2 | 0.4 | g |
| 50% DMSO water solution | 100 | ml. |

The ethanol extract as obtained in Example 2 was dissolved in 100 ml 50% DMSO water solution to obtain a working solution.

### EXAMPLE 5

| | |
|---|---|
| Dihydrotanshinone I | 0.1 g |
| Corn starch | 0.5 g |
| Lactose | 1.87 g |
| Magnesium stearate | 0.03 g |

Dihydrotanshinone I, corn starch and lactose were uniformly mixed. To the mixture a little water was added. Resulting materials was filtered and dried. Magnesium stearate was added to the mixture and uniformly mixed. The resultant was pelleted by a pelletizer. Each pellet weighs 250 mg and comprises 10 mg of active component.

Bioactivity Test

Ellman Assay

The screening for modest cholinesterease inhibitors by the method of Ellman assay was used. Human acetylcholinesterase (hAChE) in buffer was added to the test chemicals/TCM extract followed by the addition of acetylthiocholine (ATCI) substrates and Ellman's reagent, (5′5-dithio-bis-(2-nitrobenzoate), DTNB). The AChE inhibitory activity was monitored by the release of thiocholine which was then reacted with DTNB to form a bright yellow product. The optical densities of the color product formed was spectrophotometrically determined.

Both the aqueous extract obtained in Example 1 and the ethanol extract obtained in Example 2 were dissolved in 20 mg/mL 50% DMSO and later diluted in concentration 10, 20, 50, 80, 100 μg/mL for Eliman assay.

Ellman assay was preformed on a 96 well-plate as follows: a predetermined amount of aqueous or ethanol extract (as described above) in assay buffer (100 mM sodium phosphate buffer, pH 7.4) was mixed with 0.1 unit of hAChE (commercially obtained from Sigma.) After a short pre-incubation (i.e. 10 minutes), equal amount of 12.5 mM of acetylthiocholine (ATCI) was mixed with 10 mM (5′5-dithio-bis-(2-nitrobenzoate), DTNB) and the mixture was added to a well. After 10 minutes, the optical densities were measured in a 96-well plate reader at 415 nm. Optical densities was inversely proportional to the inhibiting activity.

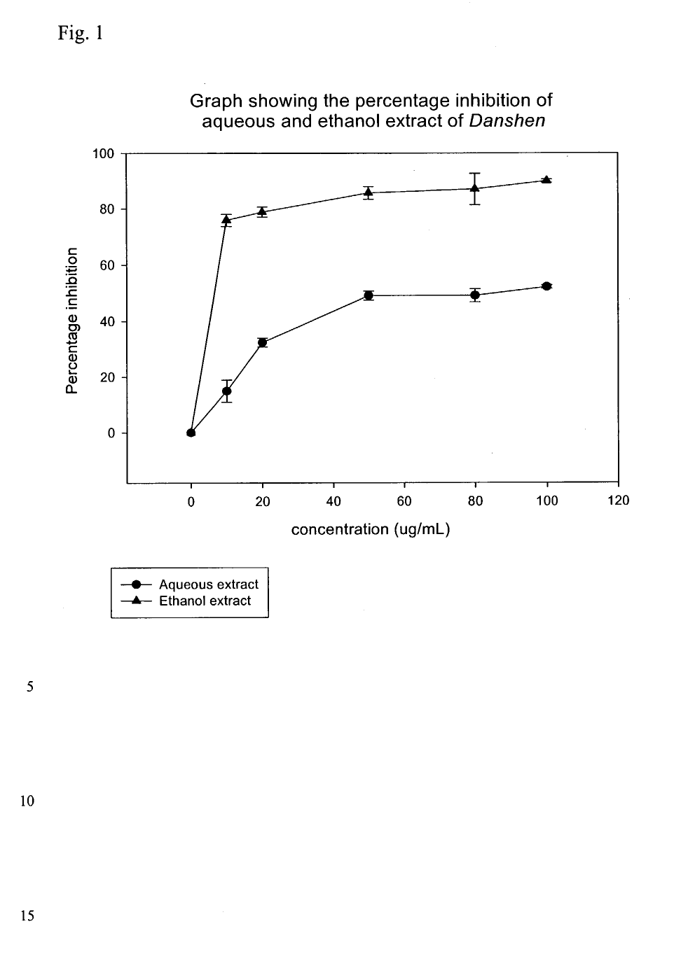

The result of the test was given in FIG. 1. The aqueous extract of root of Salvia miltiorrhiza showed a modest hAChE inhibition while the ethanol extract inhibited hAChE activity by more than 80% at 50 μg/mL.

The anti-cholinesterase activities of selected active components of Salvia miltiorrhiza were also tested. They are protocatechualdehyde (MW=138.12), tanshinone IIA (MW=294), tanshinone I (MW=276), cryptotanshinone (MW=296), salvianic acid B (MW=718) and dihydrotanshinone I (MW=278). All of them are obtained from the (National Institute for the Control of Pharmaceutical and Biological Products, State Drug Administration, P.R. China,

[symbol]

[symbol]

Selected active components were dissolved in 50% DMSO. 100% DMSO was not recommended in this assay as it lowers the enzyme vitality.

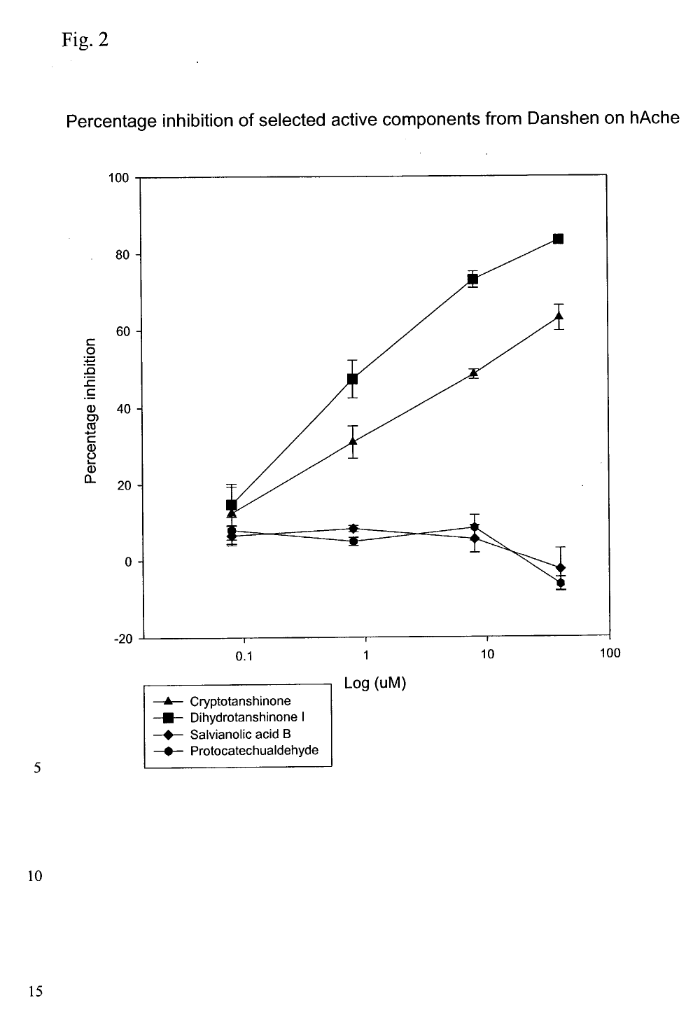

Ellman assay was performed on the selected active components, protocatechualdehyde (MW=138.12), cryptotanshinone (MW=296), salvianic acid B (MW=718) and dihydrotanshinone I (MW=278). 10-fold dilution was made with highest concentration at 40 μM. (FIG. 2)

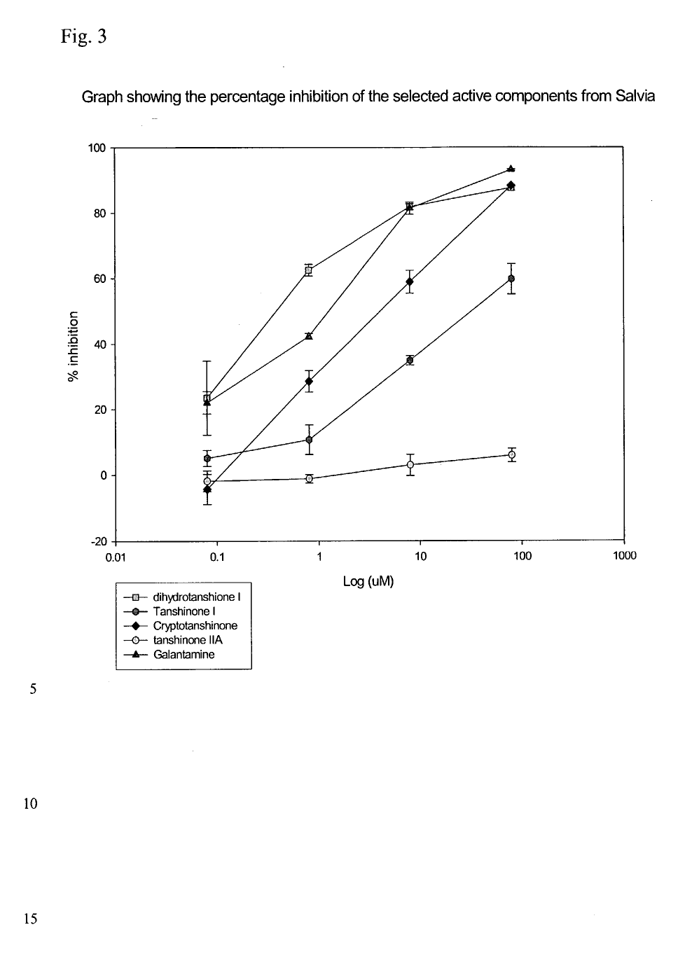

Ellman assay was preformed on the selected active components, cryptotanshinone (MW=296), tanshinone I (MW=276), tanshinone IIA (MW=294), dihydrotanshinone I (MW=278) and galanthamine (MW=368.3) (as control). 10-fold dilution was made with the highest concentration at 80 μM. (FIG. 3)

Galanthamine is an FDA approved drug for treating Alzheimer's disease.

Among the chemicals tested, only cryptotanshinone and dihydrotanshinone I showed strong AChE inhibition with IC50 at about 5-8 μM and 0.5-0.8 μM, respectively (FIG. 2 and 3 ). Tanshinone I, on the only hand, showed a modest AChE inihibiton with an IC50 about 70 μM. The structurally related diterpenoids protcatechualdehyde, tanshinone IIA, salvianic acid B had little effect on AChE inihibition.

In order to test the specificity of cryptotanshinone and dihydrotanshinone I towards hAChE, butrylcholinesterase (hBChE) instead of hAChE was used in Ellman assay. hBChE is concentrated in red blood cell and it is named as pseudoesterase as it has very homology as hAChE. Non-specific inhibitor binding to hBChE may give serious cholinergic side-effect.

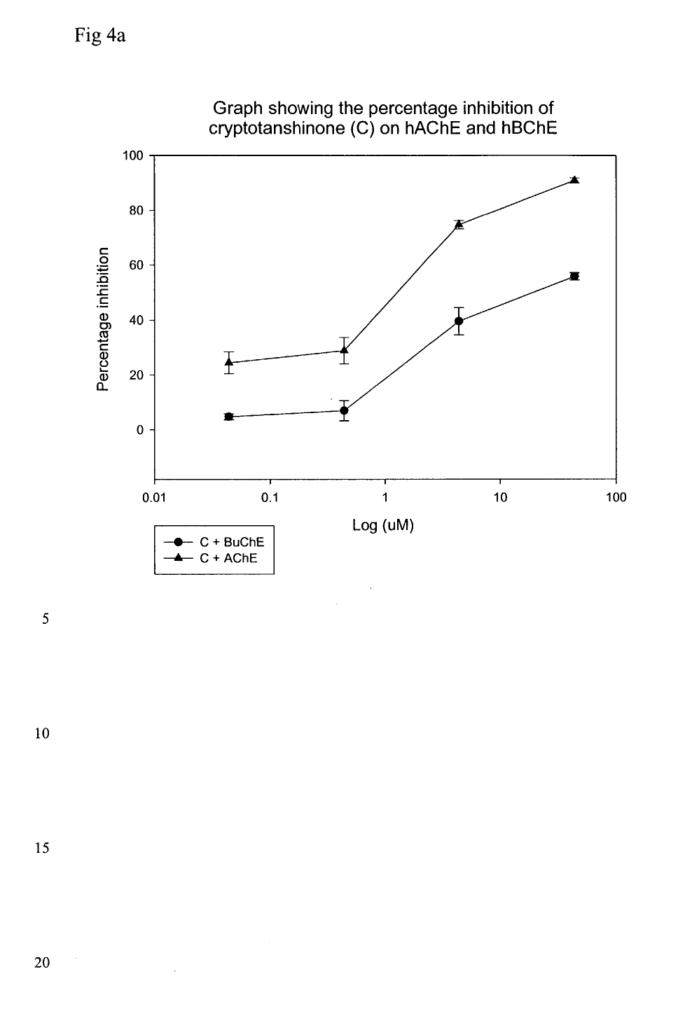

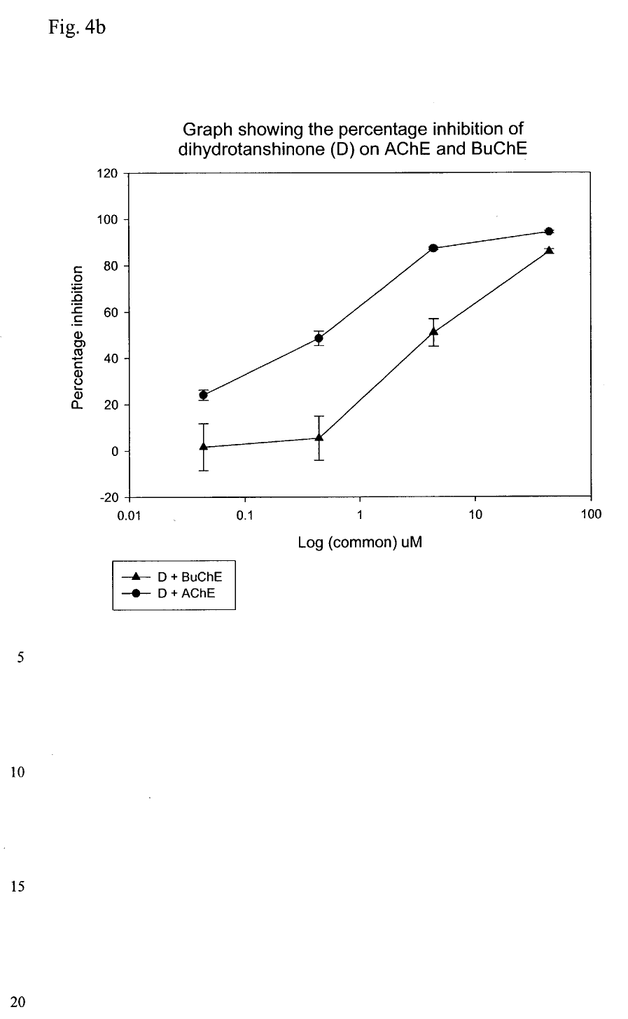

Ellman assay was preformed as mentioned, except that hBChE (commercially obtained from Sigma.) was used. Ellman assay using hAChE was also preformed in parallel for comparison. Both cryptotanshinone and dihydrotanshinone I showed higher selectivity for AChE than BChE (FIGS. 4a and 4b).

MTT Assay

MTT assay was used to check for the toxicity of the selected primary hAChE inhibitors. Since only metabolic active cell can cleave the tetrazolium salt MTT and form a formazen dye. The O.D. measure at 540 nm is directly proportional to the number of viable cell.

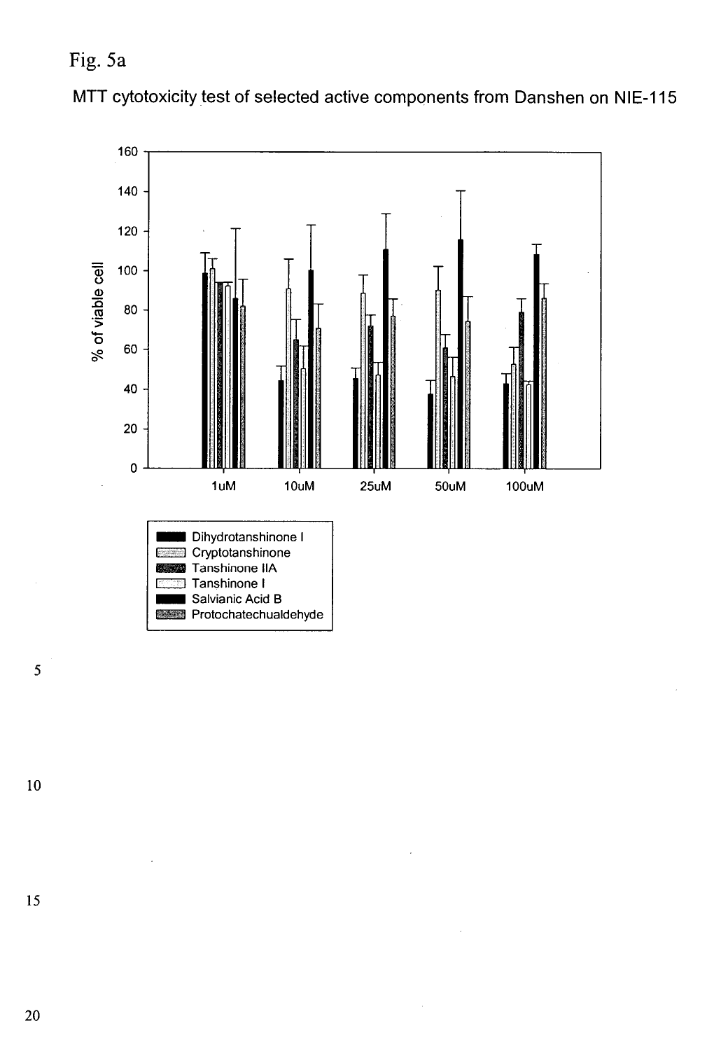

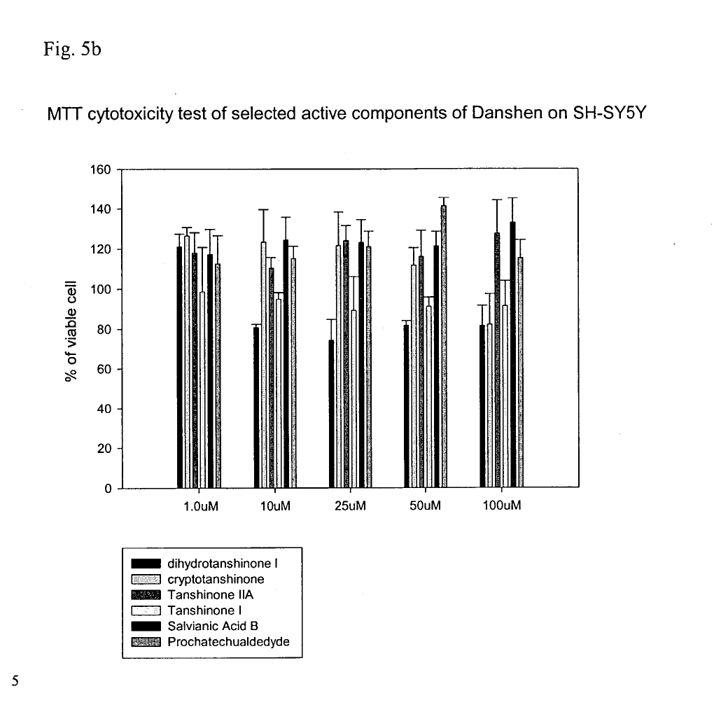

No significant cytotoxicity of cryptotashinone was found on neuronal cell line-SHSY5Y and mouse neuronal cell line N1E-115 from 1-50 μM (FIGS. 5a & 5b) and 1 μM for dihydrotanshinone I and tanshinone I.

Decrease in cytotoxic effect in cell by Tanshinone II-A was reported which was suspected through the scavenging lipid free radicals. (Zhao B. L., Jiang W, Zhao Y., Hou J. W., Xin W. J., Scavenging effects of salvia miltiorrhiza on free radicals and its protection for myocardial mitochondrial membranes from ischemia-reperfusion injury. Biochem Mol Biol Int. 1996 May, 38(6):1171-82.)

Although this invention has been described above, variations and modification of the invention will be obvious to those skilled in the art from the foregoing detailed description of the invention. It is understood that all of these variations, modifications and equivalence thereof should be included with the scope of the appended claims.

We claim:

1. A pharmaceutical composition for treating diseases or disorders associated with deficit of acetylcholine comprising a therapeutically effective amount of an active component selected from a compound of cryptotanshinone, dihydrotanshinone I, tanshinone I, or a pharmaceutically acceptable salt thereof, and a mixture of the compounds or their pharmaceutically acceptable salts or both, and a pharmaceutically acceptable carrier.

2. A pharmaceutical composition according to claim 1 comprising the active component of 0.01-99.99% by weight.

3. A pharmaceutical composition according to claim 1, wherein the active component is cryptotanshinone, dihydrotanshinone I, or tanshinone I.

4. A pharmaceutical composition according to claim 1, wherein the active component comes from an extract of Salvia genus.

5. A pharmaceutical composition according to claim 4, wherein the Salvia genus is selected from Salvia miltiorrhiza, Salvia lavandulaefolia, and Salvia divinorum and Salvia officinalis.

6. A pharmaceutical composition according to claim 5, wherein the Salvia miltiorrhiza is the root thereof.

7. A pharmaceutical composition according to claim 4, wherein the extract is an aqueous extract or ethanol extract.

8. A pharmaceutical composition according to claim 6, wherein the extract is an aqueous extract or ethanol extract.

9. A pharmaceutical composition according to claim 1, wherein the diseases include those of cognitive impairment.

10. A pharmaceutical composition according to claim 9, wherein the cognitive impairment includes presenile dementia, insomnia and Alzheimer's disease.

11. A pharmaceutical composition according to claim 1, wherein the disorders include attention defective disorder, attention defective disorder, vascular dementia, rapid eye movement behavior disorder (RBD).

12. A method for treating diseases or disorders associated with associated with deficit of acetylcholine comprising administrating to a patient a therapeutically effective amount of an active component selected from a compound of cryptotanshinone, dihydrotanshinone I, tanshinone I, or a pharmaceutically acceptable salt thereof, and a mixture of the compounds or their pharmaceutically acceptable salts or both.

13. A method according to claim 12, wherein the effective amount is from about 1 mg to 100 mg in a daily dose.

14. A method according to claim 12, wherein the component is cryptotanshinone, dihyrotanshinone I, or tanshinone I.

15. A method according to claim 12, wherein the active component comes from an extract of Salvia genus.

16. A method according to claim 15, wherein the Salvia genus is selected from Salvia miltiorrhiza, Salvia lavandulaefolia, Salvia divinorum and Salvia officinalis.

17. A method according to claim 16, wherein the Salvia miltiorrhiza is the root thereof.

18. A method according to claim 15, wherein the extract is an aqueous extract or ethanol extract.

19. A method according to claim 17, wherein the extract is an aqueous extract or ethanol extract.

20. A method according to claim 12, wherein the diseases include those of cognitive impairment.

21. A method according to claim 20, wherein the cognitive impairment includes presenile dementia, insomnia and Alzheimer's disease.

22. A method according to claim 12, wherein the disorders include attention defective disorder, attention defective disorder, vascular dementia, rapid eye movement behavior disorder (RBD).

23. A use of a compound of cryptotanshinone, dihydrotanshinone I, tanshinone I, or a pharmaceutically acceptable salt thereof, or a mixture of the compounds or their pharmaceutically acceptable salts or both in preparing a medicament for treating diseases or disorders associated with deficit of acetylcholine.
